SEQUENCE LISTING (5 sequences)

SEQ ID NO: 1 - protein, 214 aa, Artificial Sequence
  GSTP1
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLZHIBDLLLIHEVLAPGCLDAFPLLSAYVGRLSARPKLKAFLASPEYVNLPINGNGKQ

SEQ ID NO: 2 - protein, 68 aa, Artificial Sequence
  MT3
MDPETCPCPSGGSCTCADSCKCEGCKCTSCKKSCCSCCPAECEKCAKDCVCKGGEAAEAEAEKCSCCQ

SEQ ID NO: 3 - protein, 5 aa, Artificial Sequence
  LINKER
GGGGS

SEQ ID NO: 4 - protein, 283 aa, Artificial Sequence
  GSTP1-MT3
MDPETCPCPSGGSCTCADSCKCEGCKCTSCKKSCCSCCPAECEKCAKDCVCKGGEAAEAEAEKCSCCQGGGGSMPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 5 - DNA, 849 bp, Artificial Sequence
  GSTP1-MT3
atggaccccgagacctgcccctgtcccagcggaggaagctgcacctgcgccgactcctgcaagtgcgagggctgcaagtgcaccagctgcaagaagagctgctgcagctgctgccccgccgaatgcgaaaaatgtgccaaggactgcgtgtgtaaggggggcgaggccgccgaggctgaggctgagaagtgctcctgctgccaaggcggaggcggcagcatgcccccttataccgtggtgtacttccccgtgagaggcagatgcgccgccctgagaatgctgctggccgaccaaggccaaagttggaaggaggaggtggtcacagtggagacatggcaggagggcagtctgaaggcttcctgtctgtatggccagctgcccaaattccaagacggggatctgaccctgtaccagagcaacaccatactgagacatctgggccggacactgggtctctatgggaaggatcagcaggaggccgccctggtggacatggtcaacgacggagtggaggacctgagatgcaagtacatcagcctgatctacacaaactacgaggctggcaaagatgattacgtgaaagcactgcccggacagctgaaacctttcgagaccctgctgtctcagaaccagggcggcaagaccttcatcgtgggcgaccagatcagcttcgcagattacaacctgctggacctgctgctgattcatgaggttctggcccccggctgtctcgacgccttcccactgctctctgcttacgtgggccggctgagcgccagacccaagctcaaggccttcctggcctcccccgagtacgtgaacctgcccatcaacggaaacggcaagcaa

(19) United States
(12) Patent Application Publication
(10) Pub. No.: US 2021/0100916 A1
(43) Pub. Date: Apr. 8, 2021

(54) PREPARATION AND USE OF MITOCHONDRION-TARGETING SELF-ASSEMBLED PROTEIN NANOPARTICLE

(71) Applicant: PEKING UNIVERSITY, Beijing (CN)

(72) Inventors: Jian LIN, Beijing (CN); Xinjie ZHU, Beijing (CN); Liang XU, Beijing (CN); Nuo XU, Beijing (CN); Long CHEN, Beijing (CN)

(73) Assignee: PEKING UNIVERSITY, Beijing (CN)

(21) Appl. No.: 17/050,188

(22) PCT Filed: Aug. 15, 2019
(86) PCT No.: PCT/CN2019/100706

§ 371 Date: Oct. 23, 2020

(30) Foreign Application Priority Data
Nov. 9, 2018 (CN) 201811336263.4

(51) Int. Cl. A61K 47/69; A61K 47/65; A61K 47/64

(52) U.S. Cl. CPC A61K 47/6929; A61K 47/64; A61K 47/65

(57) ABSTRACT

A mitochondrion-targeting protein nanoparticle, containing related information such as an amino acid sequence thereof, a coding nucleic acid sequence, and a vector and a host expression bacterium for the coding nucleic acid. Under the induction of metal ions, the self-assembled protein nanoparticle mentioned above can be obtained by an Escherichia coli expression system. The protein nanoparticle can be applied in cancer diagnosis and treatment. Compared with existing mitochondrion-targeting small molecules (such as TPP and MPP), the protein nanoparticle can achieve properties of tumor enrichment, mitochondrion targeting, increasing of ROS content in cells, induction of cell apoptosis, inhibiting tumor growth, etc.

### TECHNICAL FIELD

The present disclosure relates to the fields of bioengineering and medicine, particularly in the technical fields of tumor imaging and therapy as well as drug delivery. Specifically, the present disclosure relates to a novel mitochondrion-targeting protein nanoparticle (GSTP1-MT3), which has related properties such as mitochondrion targeting, causing mitochondrial functional dysfunction, accumulating around tumors and inhibiting tumor growth, etc.

### BACKGROUND

Cancer is a worldwide problem that needs to be solved urgently. According to statistics from the World Health Organization, nearly 14 million people die from cancer each year. Breast cancer is the second most common cancer for women, of which the morbidity rates are less than skin diseases. According to statistics from the US Department of Health, nearly 260,000 women will die from breast cancer each year. Therefore, breast cancer therapies have attracted more and more attentions.

However, patients usually realized their own symptoms in a late stage of cancer. Meanwhile, many cancer patients will have tumor-drug resistance for traditional chemotherapy drugs. It takes great challenges for cancer therapy. Mitochondria which served as main energy currency of the cell plays vital roles in maintaining cell growth and metabolism. Since mitochondrial functional dysfunction will cause irreversible damage to cells, more and more people are exploring cancer therapeutic options based on mitochondrial functional dysfunction. Recently, it has been discovered that mitochondrial functional dysfunction could overcome tumor drug resistance to a certain extent. As so far, mitochondria-targeting drugs are mainly small molecules such as triphenylphosphine analogues (TPP) and mitochondrial penetrating peptides (MMP). However, these small molecules have some drawbacks such as short half-life time and unspecific accumulate around tumors, which greatly limit their application in cancer therapies.

### SUMMARY

To effectively solve the above problems, the present disclosure provides a novel mitochondrion-targeting protein nanoparticle (GSTP1-MT3), which can be obtained by fusion expression using Escherichia coli as host bacteria then induced by metal ions. It was found that GSTP1-MT3(Co²⁺) protein nanoparticles can be accumulated at the tumor site in 4T1 xenograft Balb/c model and can quickly target the mitochondria, then decrease mitochondrial membrane potential and induce ROS production, which can ultimately inhibit tumor growth effectively. At the same time, GSTP1-MT3(Co²⁺) which conjugated with paclitaxel through chemical linker can not only improve the water solubility of paclitaxel, but also can greatly prolong the survival time of mice, which provides a broad prospect for clinical breast cancer therapy.

In a first hand, the present disclosure provides a novel mitochondrion-targeting self-assembled protein nanoparticle, characterized in that the protein nanoparticle is formed by self-assembly of protein monomers, and the protein monomer include a fusion protein composed of metallothionein, linker peptide, and glutathione sulfur transferase connected in sequence from amino terminus to carboxy-terminus, wherein an amino acid sequence of the linker peptide is shown in SEQ ID NO: 3.

The protein nanoparticle of the present disclosure is characterized in that the glutathione sulfur transferase is GSTP1 and an amino acid sequence of the GSTP1 is shown in SEQ ID NO: 1.

The protein nanoparticle of the present disclosure is characterized in that the metallothionein is preferably MT3, and an amino acid sequence of the MT3 is shown in SEQ ID NO: 2.

The self-assembled protein nanoparticle of the present disclosure is characterized in that: the protein nanoparticle is a fusion protein which is induced by metal ions, and an amino acid sequence thereof is shown in SEQ ID NO: 4. The metal ions include Cd²⁺, Gd³⁺, Cr³⁺, Ni²⁺, Fe²⁺, Mn²⁺, and Co²⁺.

The protein nanoparticle of the present disclosure is characterized in that: the glutathione sulfur transferase is located at the C-terminus of the fusion protein.

In a second aspect, the present disclosure also provides a nucleic acid encoding the fusion protein in the protein nanoparticle, and a nucleotide sequence of the nucleic acid is shown in SEQ ID NO: 5.

In a third aspect, the present disclosure also provides a construct including the nucleic acid, and the construct includes an expression cassette and a vector.

The vector of the present disclosure includes a prokaryotic expression vector, and the expression vector includes a promoter, a terminator and other regulatory elements.

In a fourth aspect, the present disclosure also provides a host expression bacteria containing the nucleic acid and the construct.

The host expression bacteria of the present disclosure include prokaryotic host expression bacteria, and the prokaryotic host expression bacteria is BL21(DE3).

In a fifth aspect, the present disclosure also provides the application of the mitochondrion-targeting protein nanoparticle in cancer diagnosis.

The protein nanoparticle can also conjugate with an active substance, and the active substance is a diagnostic reagent.

The protein nanoparticles can be accumulated in tumor regions. The diagnostic reagents include fluorescent groups, isotopes, MRI contrast agents, radioisotopes and other substances.

In a sixth aspect, the present disclosure also provides the application of the mitochondrion-targeting protein nanoparticle in tumor therapies.

The mitochondrion-targeting protein nanoparticle can inhibit tumor growth.

The protein nanoparticle is conjugated with an active substance, the active substance is a tumor treatment drug, the tumor therapeutic drug is paclitaxel, and the tumor is breast cancer.

In a seventh aspect, the present disclosure also provides a method for preparing the novel mitochondrion-targeting self-assembled protein nanoparticle.

The method for preparing the mitochondrion-targeting novel protein nanoparticle is performed under the induction of 0.3 mM metal ions, which can chelate with the amino groups or sulfhydryl groups of proteins, protein monomers are self-assembled to form protein nanoparticles.

The method for preparing the mitochondrion-targeting novel protein nanoparticle, wherein the metal ions include Cd²⁺, Gd³⁺, Cr³⁺, Ni²⁺, Fe²⁺, Mn²⁺, and Co²⁺.

The method for preparing the mitochondrion-targeting novel protein nanoparticle further includes the step of purifying the protein nanoparticles formed by self-assembly using a GST purification column.

In an eighth aspect, the present disclosure provides a protein nanoparticle that is capable of significantly improving the water solubility of paclitaxel.

The combined use of the protein nanoparticles and paclitaxel significantly prolongs the survival time of 4T1 breast cancer mice.

Comparing with the prior art, the technical scheme of the present disclosure has the following advantages:

Firstly, for the first time, it is reported that self-assembled protein nanoparticles induced by metal ion can quickly target mitochondria so that mitochondrial changes can be monitored in real time. As compared with other mitochondrion-targeting small molecules, the protein nanoparticle has better biocompatibility and water solubility, and its targeting speed is fast, which can better reflect the real situation of mitochondria in real time.

Secondly, the protein nanoparticle can decrease mitochondrial membrane potential and increase ROS production in cells, then cause cell apoptosis.

Thirdly, tumor-drug resistance is the main obstacle that restrict the cancer therapeutic efficacy for traditional drugs. As an intracellular ATP main energy currency of the cell which plays an important role in cell growth and division. Since the damage of mitochondria is irreversible, the mitochondrial functional dysfunction may overcome the problems effectively. The protein nanoparticles may play an important role in overcoming tumor-drug resistance.

Finally, comparing with other mitochondrion-targeting small molecules, the protein can accumulate in tumor regions effectively, which can reduce the damage of drugs to other organs, thereby reducing the damage to normal tissues.

### BRIEF DESCRIPTION OF THE DRAWINGS

Upon reading a detailed description of the preferred embodiments below, various other advantages and benefits will become clear to those skilled in the art. The drawings are only for the purpose of illustrating the preferred embodiments and should not be considered as limiting the present disclosure.

FIG. 1: Amino acid sequence composition of GSTP1-MT3 mitochondrion-targeting protein nanoparticle;

FIG. 2: Characterization of GSTP1-MT3 protein nanoparticles;

A: Protein expression induced by Co²⁺ and Ni²⁺ respectively;

B: Protein expression levels under the induction of 1 mmol/L of different metal ions;

C: Determination of GSTP1-MT3(Co²⁺) particle size;

D: The conformation changes of GSTP1-MT3(Co²⁺) protein nanoparticles in different pH condition;

E-F: The morphology of GSTP1-MT3(Co²⁺) protein nanoparticles detecting by SEM (E) and TEM (F);

FIG. 3: Expression levels under different Co²⁺ concentrations;

FIG. 4: GSTP1-MT3(Co²⁺) targeted mitochondria;

FIG. 5: Pearson's co-localization efficiency of mitochondria;

FIG. 6: GSTP1-MT3 protein nanoparticles can induce ROS production in cells;

FIG. 7: Determination of ROS content in cells;

FIG. 8: Enrichment of GSTP1-MT3(Co²⁺) around the tumors;

FIG. 9: Inhibition of tumor growth by GSTP1-MT3(Co²⁺);

A: Change of mouse body weight, with the arrows representing the time points of drug administration;

B: Determination of tumor size in mice, with the arrows represents the time points of drug administration;

C: Photographs of mice (upper row) and tumors (bottom row) taken on day 21;

D: Tumor weights measured on day 21;

FIG. 10: Prolonging the survival period of 4T1 breast cancer mice by GSTP1-MT3(Co²⁺) coupled with PTX (paclitaxel);

FIG. 11: Schematic diagram of the anti-tumor mechanism of GSTP1-MT3 protein nanoparticles in vivo.

### DETAILED DESCRIPTION

Hereinafter, exemplary embodiments of the present disclosure will be described in more detail with reference to the accompanying drawings. Although the exemplary embodiments of the present disclosure are shown in the drawings, it should be understood that the present disclosure can be implemented in various forms and should not be limited by the embodiments set forth herein. Rather, these embodiments are provided to enable a more thorough understanding of the present disclosure and to fully convey the scope of the present disclosure to those skilled in the art.

According to the embodiments of the present disclosure, the following examples are proposed.

### EXAMPLE 1

### Construction of Amino Acid Sequence of GSTP1-MT3 Mitochondrion-Targeting Protein Nanoparticle

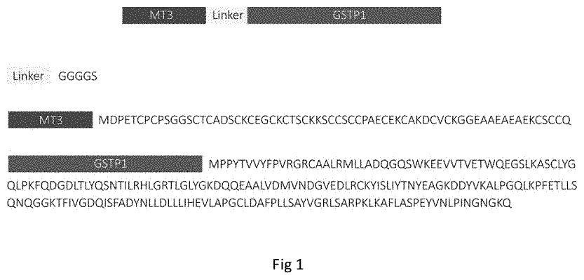

Based on earlier experimental studies, the following mitochondrion-targeting protein nanoparticle expression vector was constructed. As shown in FIG. 1, GSTP1-MT3 is mainly composed of GSTP1 and MT3, wherein MT3 is at the N-terminal of the amino acid sequence, GSTP1 is at the C-terminal of the amino acid sequence, and MT3 and GSTP1 are coupled through the GGGGS sequence.

GSTP1 amino acid sequence:

(SEQ ID NO: 1)
MPPYTVVYFPVRGRCAALRMLLADQGQSWKEEVVTVETWQEGSLKASCLY
GQLPKFQDGDLTLYQSNTILRHLGRTLGLYGKDQQEAALVDMVNDGVEDL
RCKYISLIYTNYEAGKDDYVKALPGQLKPFETLLSQNQGGKTFIVGDQIS
FADYNLLDLLLIHEVLAPGCLDAFPLLSAYVGRLSARPKLKAFLASPEYV
NLPINGNGKQ;

MT3 amino acid sequence:

(SEQ ID NO: 2)
MDPETCPCPSGGSCTCADSCKCEGCKCTSCKKSCCSCCPAECEKCAKDCV
CKGGEAAEAEAEKCSCCQ;

the linker between GSTP1 and MT3:

(SEQ ID NO: 3)
GGGGS.

The complete amino acid sequence of GSTP1-MT3 is shown in SEQ ID NO: 4, its molecular weight is 30.566 kDa, and its isoelectric point (pI) is 5.14; the encoding nucleotide sequence of GSTP1-MT3 is shown in SEQ ID NO: 5.

### EXAMPLE 2

### Construction of GSTP1-MT3 Protein Nanoparticle Vector

In order to facilitate the subsequent expression of GSTP1-MT3 protein nanoparticles in recombinant cells, a prokaryotic expression vector was constructed, wherein pET-28a(+) was selected as the prokaryotic expression vector, restriction sites HindIII and Ndel thereon were used to connect the nucleotide sequence encoding GSTP1-MT3 shown in SEQ ID NO: 5 into pET-28a(+), and a recombinant expression vector pET-28a(+)-GSTP1-MT3 was successfully obtained after restriction enzyme digestion, electrophoresis and monoclonal sequencing.

### EXAMPLE 3

### Recombinant Expression of GSTP1-MT3 Protein Nanoparticles

Escherichia coli is used as the host bacteria for recombinant expression, and the specific expression method is as follows:

1. Plasmid Transformation

2 μL of 42 ng/μL pET-28a(+)-GSTP1-MT3 plasmid was taken and added into 20 μL of BL21(DE3) competent cells, was pre-mixed on ice for 15-30 minutes, was then heated in a 42° C. water bath pot for 90 seconds, and was then placed on ice for another 10 minutes. 800 μL of resistance-free LB culture medium was added, incubated at 37° C., 220 rpm for 1 hour, and then centrifuged at 3500 rpm for 10 minutes; 600 μL of supernatant was removed, and the remaining 200 μL of bacterial solution was mixed well for later use.

2. Resistance Screening

The remaining 200 μL of bacterial solution in step 1 was added to an agarose plate containing kanamycin. After being incubated at 37° C. for 2 hours, the plate was inverted and incubated overnight.

3. Monoclonal Selection

A single clone was selected and added to 10 mL of LB culture medium containing kanamycin, and incubated at 37° C., 220 rpm for 10 hours, and the solution gradually became turbid.

4. Induced Protein Expression

10 mL of the bacterial solution in step 3 was added to 1 L of LB culture medium containing kanamycin. After being incubated at 37° C., 22 0rpm for 4 hours, 1 mL of 0.1 mol/L IPTG (the final concentration in the culture medium is 0.1 mmol/L) and 1 mmol/L different metal ions (Cd²⁺, Gd³⁺, Cr³⁺, Ni²⁺, Zn²⁺, Fe²⁺, Mn²⁺, Co²⁺) were added respectively to continue to induce expression overnight. The solution was centrifuged at 4° C., 4000 rpm for 20 minutes, the supernatant was removed, and 20 mL of GST resuspension solution (pH=8.0, 50 mM Tris/HC1, 100 mM NaCl, 60 mM β-Mercaptoethanol) was added for ultrasonic crushing (30% power, SCIENTZ, JY 92-IIN). The solution was ultracentrifugated at 4° C., 12000 rpm, and the supernatant was collected and filtered with a 0.22 μm filter membrane, waiting for further purification.

5. Protein Purification

The AKTA purification system was used for purification. First, a 5 times (5V) column volume of PBS was used for equilibration, then the AKTA was used to combine the supernatant in step 4 onto the GST column. The 5 V column volume of PBS continued to be used to wash until the baseline was stable. The GST solution (pH=8.0, 10 mmol/L GSH, 50 mM Tris/HC1, 100 mM NaCl, 60 mM β-Mercaptoethanol) was used for elution and collection. The collected protein was filtered with a 10 kDa ultrafiltration tube to remove GSH from the protein, and finally a 0.22 μm filter membrane was used for filtration. Short-term preservation was performed at 4° C. (if long-term preservation at −80° C. was performed, the preservation should be in 10% glycerol).

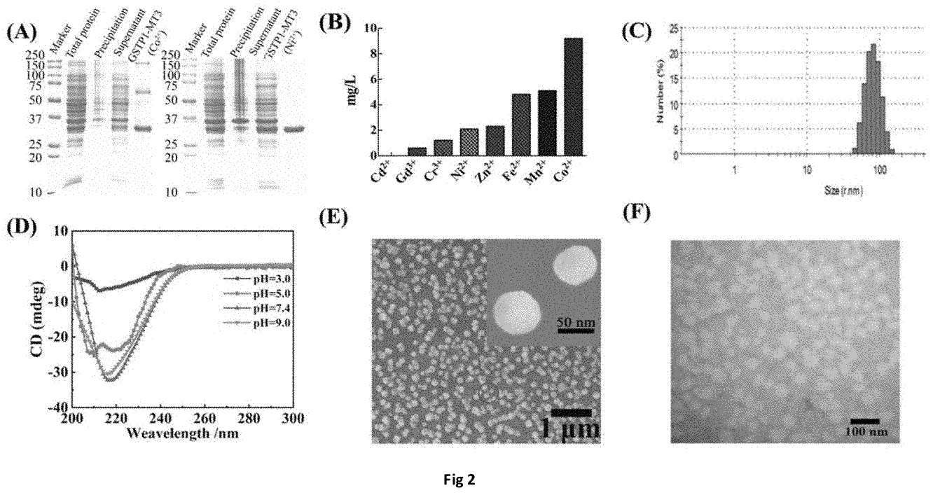

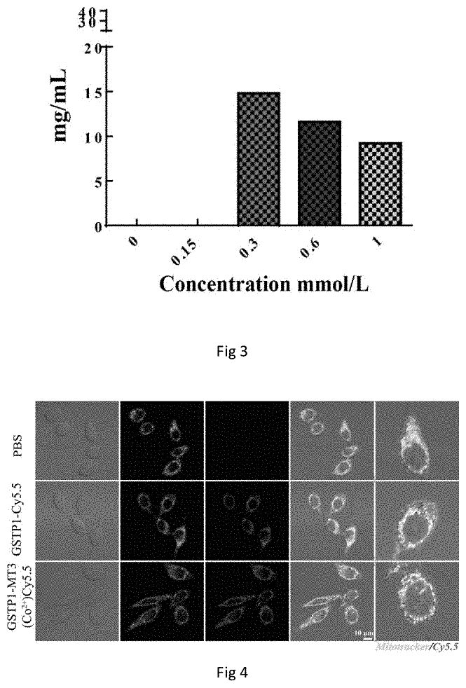

As shown in FIG. 2, GSTP1-MT3 with a molecular weight of approximately 30 kDa can be obtained by either Co²⁺ or Ni²⁺ induction (FIG. 2A); and 1 mmol/L of different metal ions (Cd²⁺, Gd³⁺, Cr³⁺, Ni²⁺, Zn²⁺, Fe²⁺, Mn²⁺, Co²⁺) can each be used to induce GSTP1-MT3 protein nanoparticles, wherein Co²⁺ has the strongest inducing ability (FIG. 2B). Therefore, in the following, expression levels under different Co²⁺ concentrations have been studied, and the results showed that the most suitable induction concentration of Co²⁺ is 0.3 mmol/L (FIG. 3) (unless specifically noted, the induction concentration of each metal ion is 0.3 mmol/L). The function of the protein nanoparticles induced by Co²⁺ was analyzed; FIG. 2D shows the secondary structure of GSTP1-MT3 protein nanoparticles in different pHs; It can be seen from FIG. 2C and FIGS. 2E-2F that after the metal ion induction, GSTP1-MT3 forms nanoparticles with uniform size distribution.

### EXAMPLE 4

### GSTP1-MT3 Labeled Cy5.5 Fluorescence

Taking the GST-MT3 (Co²⁺) obtained in Example 3 as an example, 500 μL of 100 μM GST-MT3 (Co²⁺) was added into a Tris/HC1 buffer at pH=9.0; after sufficient mixing, 6.7 μL of 15 mM Cy5.5-NHS (Cy5.5-NHS was dissolved in DMSO) is added, mixed well quickly, and put into a mixer at 25° C., 1000 rpm for reaction overnight (the whole process was protected from light). A desalting column was used to remove the unreacted Cy5.5-NHS. Finally, a 0.22 pm filter membrane was used for filtration, and the product after the reaction was labeled as GSTP1-MT3(Co²⁺)-Cy5.5 and preserved at 4° C. to avoid light.

### EXAMPLE 5

### GSTP1-MT3 Targeted Mitochondria

Taking U87MG cell line as an example, 5×10⁴ U87MG cells were added to a confocal dish and incubated overnight at 37° C. and 5% CO₂; GSTP1-MT3(Co²⁺)-Cy5.5 prepared in Example 4 was added to it (the final concentration was 6 μmo/L), then washed with PBS for three times; finally, Mitotracker® Green FM (the final concentration was 2 μmol/L) was added, incubated again at 37° C., 5% CO₂ for 30 minutes, and finally washed with PBS for three times; confocal imaging was performed, and Image J was used to calculate Pearson's co-localization efficiency.

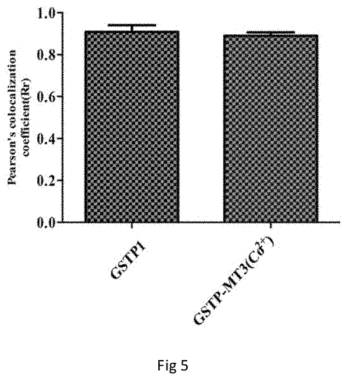

As shown in FIG. 4 and FIG. 5, the protein nanoparticles can be well positioned to the mitochondria, and GSTP1 plays a key role in mitochondria targeting.

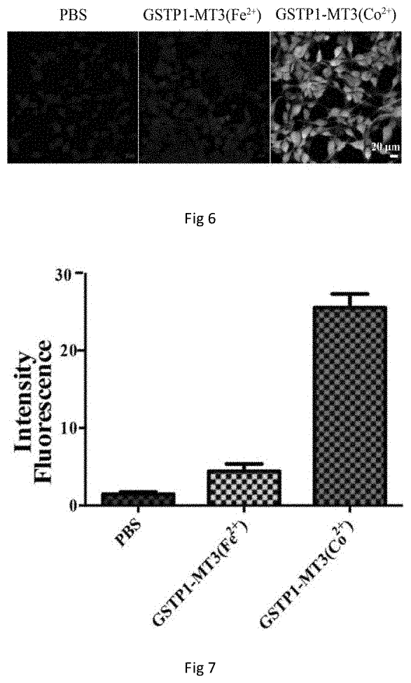

Taking U87MG cell line as an example, 5×10⁴ U87MG cells were added to a confocal dish and incubated overnight at 37° C. and 5% CO₂; GSTP1-MT3(Fe²⁺) and GSTP1-MT3(Co²⁺) prepared in Example 3 were added to it (the final concentration was 6 μmo/L) and incubated again for 24 hours, and then washed with PBS for three times; then, 10 μmol/L DCFH-DA (ROS probe) was added, incubated at 37° C. for 30 minutes, and washed with PBS for three times; finally, confocal imaging was performed, and statistics was performed on the ROS content. The results shown in FIGS. 6 and 7 show that GSTP1-MT3 (Co²⁺) can significantly increase the ROS content in cells.

### EXAMPLE 6

### Enrichment of GSTP1-MT3 (Co²⁺) in Tumors

Taking the breast cancer of Balb/c mice as an example, 3×10⁶ 4T1 (stable transfected Luciferase) breast cancer cells were inoculated into Balb/c mice; when the tumor grew to 150 mm³, 100 μL of 30 μmol/L GSTP1-MT3(Co²⁺)-Cy5.5 was injected through the tail vein; the fluorescence intensity was observed at different time points, and then Luciferase substrate was injected through the abdominal cavity to observe the co-localization of the tumor.

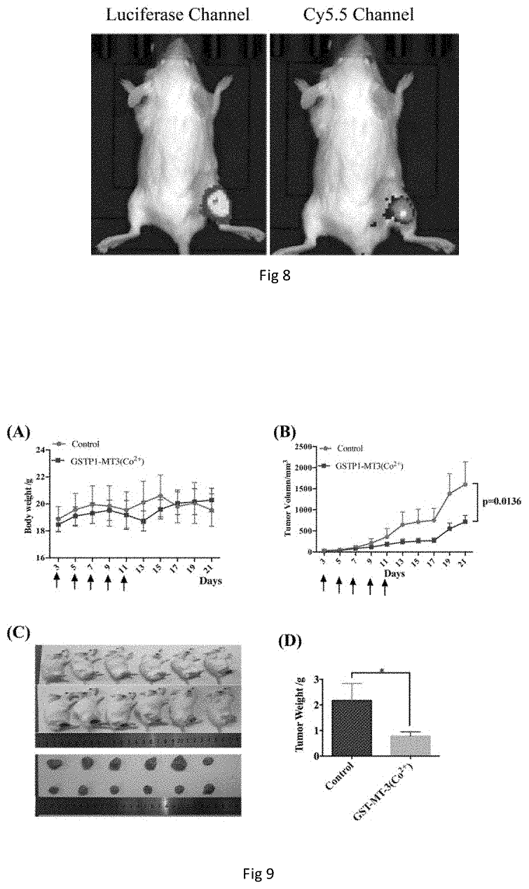

The results are shown in FIG. 8. Cy5.5 fluorescence and Luciferase bioluminescence can overlap well together, indicating that GSTP1-MT3(Co²⁺)-Cy5.5 can be well enriched at the tumor site.

### EXAMPLE 7

### GSTP1-MT3(Co²⁺) Treatment of Breast Cancer

Taking the breast cancer of Balb/c mice as an example, 3×10⁶ 4T1 (stable transfected Luciferase) breast cancer cells were inoculated into Balb/c mice; when the tumor grew to 50-70 mm³, 100 μL of 30 pmol/L GSTP1-MT3(Co²⁺) was injected through the tail vein as the experimental group, and 100μL of PBS was injected through the tail vein as the control group. The injection was performed once every two days, that is, the tail vein injection was performed on the 3^{rd}, 5^{th}, 7^{th}, 9^{th} and 11^{th} day of the experiment, respectively. A total of 5 injections were given. The weights of the mice and the size of the tumor were recorded at different time points.

As shown in FIG. 6, the body weights of the mice in the experimental group and the control group were not much different, indicating that GSTP1-MT3(Co²⁺) had no obvious toxic or side effects on mice; in terms of the tumor volume and tumor weight, the tumor volumes of the mice in the experimental group and the control group were significantly different (P<0.05), and the tumor weights of the mice in the experimental group and the control group were significantly different (P<0.05), indicating that GSTP1-MT3 protein nanoparticles can significantly inhibit tumor growth.

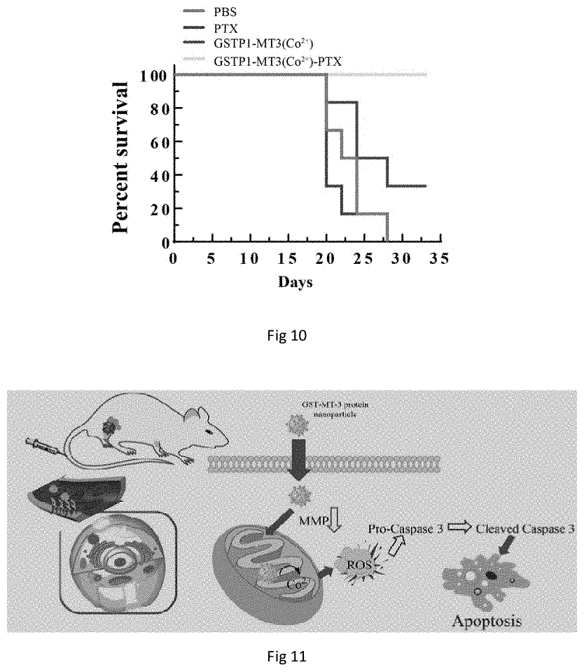

At the same time, it is found that GSTP1-MT3(Co²⁺) coupled with paclitaxel can significantly prolong the survival period of 4T1 breast cancer mice (FIG. 10). The schematic diagram of the anti-tumor mechanism of GSTP1-MT3 in vivo is shown in FIG. 11.

Described above are only specific preferred embodiments of the present disclosure, but the scope of protection of the present disclosure is not limited to this. Any change or replacement that can be easily contemplated by those skilled in the art within the technical scope disclosed in the present disclosure should be covered within the scope of protection of the present disclosure. Therefore, the scope of protection of the present disclosure shall be accorded with the scope of the claims.

1. A mitochondrion-targeting self-assembled protein nanoparticle,
wherein the protein nanoparticle is formed by self-assembly of protein monomers, and the protein monomer comprise a fusion protein composed of metallothionein, linker peptide, and glutathione sulfur transferase connected in sequence from amino-terminus to carboxy-terminus, and wherein an amino acid sequence of the linker peptide is shown in SEQ ID NO: 3.

2. The self-assembled protein nanoparticle according to claim 1, wherein the glutathione sulfur transferase is GSTP1, and an amino acid sequence of the GSTP1 is shown in SEQ ID NO: 1; and wherein the metallothionein is MT3, and an amino acid sequence of the MT3 is shown in SEQ ID NO: 2.

3. The self-assembled protein nanoparticle according to claim 1, wherein the protein nanoparticle is a fusion protein formed by metal ions induction, and an amino acid sequence thereof is shown in SEQ ID NO: 4; and wherein the metal ions comprise Cd²⁺, Gd³⁺, Cr³⁺, Ni²⁺, Fe²⁺, Mn²⁺, and Co^{2±}.

4. A method for preparing a mitochondrion-targeting self-assembled protein nanoparticle,
wherein under the induction of metal ions, through the coordination of metal ions and the amino and sulfhydryl groups on proteins, protein monomers are self-assembled to form protein nanoparticles.

5. A nucleic acid encoding the fusion protein in the protein nanoparticle according to claim 1,
wherein a nucleotide sequence of the nucleic acid is shown in SEQ ID NO: 5.

6. A nucleic acid construct, comprising the nucleic acid according to claim 5, wherein the nucleic acid construct comprises an expression cassette and a vector.

7. A prokaryotic host cell or a eukaryotic host cell, containing the nucleic acid according to claim 5.

8. Use of the mitochondrion-targeting protein nanoparticle according to claim 1 in cancer diagnosis drugs or therapeutic drugs.

9. Use of the protein nanoparticle according to claim 1 in the preparation of tumor therapeutic drugs.

10. The use according to claim 8,
wherein the protein nanoparticle can be connected with an active substance, the active substance is a diagnostic marker or tumor therapeutic drug, and the active substance mainly comprises fluorescent molecules, isotopes, MRI contrast agents, radioactive substances, anti-oxidation drugs and anticancer drugs.
